(19) United States
(12) Patent Application Publication
(10) Pub. No.: US 2022/0226599 A1
(43) Pub. Date: Jul. 21, 2022

(54) VALVE ASSEMBLY, VENTILATOR, PROCESS FOR OPERATING A VALVE ASSEMBLY, AND COMPUTER PROGRAM

(71) Applicant: Drägerwerk AG & Co. KGaA, Lübeck (DE)

(72) Inventors: Hans-Ullrich HANSMANN, Lübeck (DE); Oliver GARBRECHT, Lübeck (DE); Hendrik FISCHER, Lübeck (DE); Georg MÄNNEL, Lübeck (DE)

(21) Appl. No.: 17/572,894

(22) Filed: Jan. 11, 2022

(30) Foreign Application Priority Data
Jan. 21, 2021 (DE) 10 2021 101 207.4

(51) Int. Cl. A61M 16/20

(52) U.S. Cl. CPC A61M 16/201; A61M 16/205; A61M 16/204; A61M 16/203

(57) ABSTRACT

A valve assembly, a ventilator, a process for operating a valve assembly and a computer program are provided. The valve assembly (10; 10a; 10b), for the ventilator (100), includes an inlet (12; 12a; 12b) configured for the inflow of a ventilation gas, an outlet (14; 14a; 14b) configured for the outflow of the ventilation gas and a volume flow control device (16; 16a; 16b) for the ventilation gas between the inlet and the outlet. The volume flow control device is configured to set the volume flow of the ventilation gas in a range between shut-off and a maximum volume flow and to provide an attenuation of a volume flow change during the opening, when the volume flow of the ventilation is increased, that differs from an attenuation occurring during the closing, when the volume flow of the ventilation gas is reduced.

### CROSS REFERENCE TO RELATED APPLICATIONS

This application claims the benefit of priority under 35 U.S.C. § 119 of German Application 10 2021 101 207.4, filed Jan. 21, 2021, the entire contents of which are incorporated herein by reference.

### TECHNICAL FIELD

The present invention pertains to a valve assembly, to a ventilator, to a process for operating a valve assembly and to a computer program, especially but not exclusively to a concept for a more robust control of a volume flow in a ventilator.

### TECHNICAL BACKGROUND

Fast proportional valves are used, as a rule, for controlling mechanical ventilation or breathing assist in order to control and/or regulate volume flows to a patient (inhalation) and away from a patient (exhalation).

It is optionally also possible to use gas sources that can be modulated rapidly. These are found mostly in breathing assist and CPAP (continuous positive airway pressure) devices.

The control of the volume flows during ventilation requires high dynamics. Electromagnetic drives are usually used in this area. These components are large, heavy and require a high power input. The faster these components are to be regulated, the greater is the effect of the drawbacks. The drawbacks represent a significant factor for a cost-effective and/or mobile application.

As an alternative, the valves may also be acted on pneumatically. The above-mentioned drawbacks are largely eliminated thereby. However, this usually leads to a highly pronounced tendency to oscillations, as a consequence of which it is necessary either to greatly limit the frequency range or else the controller of the valves must be adjusted very accurately to the conditions (including to the properties of the patient).

### SUMMARY

Based on this, one object of the present invention is to create an improved concept for a volume flow regulation during the ventilation of a patient.

The object is accomplished according to a valve assembly, a ventilator, a ventilation system, a process and a computer program process according to the invention.

Exemplary embodiments are based on the discovery that rapid volume flow changes are desirable during the opening of the valves at the beginning of the breathing phases during the ventilation of a patient. A great increase in the volume flow shall take place at the beginning of the inhalation. The volume flow changes are, by contrast, slower during or by the end of the inhalation. This is also true during the exhalation, and the volume flow shall also be increased rapidly at the beginning and the changes occurring during the further course are slower. Based on this discovery, a valve assembly can be configured such that an attenuation (a dampening) of a volume flow change, which attenuation occurs during the opening, is different from an attenuation occurring during the closing. This makes possible the desired dynamics of the volume flow in the course of the breathing phases and has the effect that undesired disturbance variables are attenuated. The disturbance variables usually have a harmonic course and thus require harmonic changes of the volume flow, i.e., equally rapid changes both during the increase and during the lowering. However, since the attenuations are different during opening and during closing in exemplary embodiments, the disturbance variables are thus attenuated, as a whole.

Exemplary embodiments create a valve assembly for a ventilator with an inlet, which is configured to allow a ventilation gas to flow in, and an outlet, which is configured to allow the ventilation gas to flow out. The valve assembly further comprises a device for volume flow control for the ventilation gas between the inlet and the outlet. The volume flow control device is configured to set the volume flow of the ventilation gas in a range between shut-off and a maximum volume flow. The volume flow control device is configured such that an attenuation (a dampening) of a volume flow change during opening, when the volume flow of the ventilation gas is increased, differs from an attenuation (a dampening) occurring during closing, when the volume flow of the ventilation gas is reduced. The valve assembly can thus suppress disturbance variables in exemplary embodiments despite maintenance of the desired dynamics, so that the further effort needed in respect to these disturbance variables may decrease and in particular, the complexity of the controllers can be reduced.

The attenuation may have a limiting effect on the volume flow change rates in exemplary embodiments, so that maximum volume flow changes per unit of time are limited differently during opening and closing. As a result, disturbance variables, which are usually above the limitation at least in one frequency range, can be attenuated or reduced.

The volume flow control device may be configured, for example, such that a shortest opening time period, during which the control takes place from shut-off to the maximum volume flow, and a shortest closing time period, during which the control takes place from the maximum volume flow to the shut-off, are different from one another. This may happen in case of complete opening and closing, but also in case of changes in a middle range of the volume flow. Accordingly, the volume flow control device may also be configured such that an attenuation occurring during opening, when the control takes place from the shut-off to the maximum volume flow, is different from the attenuation occurring during closing, when the control takes place from the maximum volume flow to shut-off.

For example, the volume flow control device may have a lower attenuation during opening than during closing. This may then mean when the ventilation is carried out that the volume flow can be changed rapidly at the beginning of the respective breathing phase, but the closing operation takes place more slowly. In other words, the volume flow control device may consequently have, for example, a higher attenuation during closing than during opening.

When the ventilation is carried out, it is possible, in particular, to achieve the advantage that based on the fact that the closing process is deliberately configured to be slower, the control, i.e., the parameters of an analog or digital controller can be configured with a correspondingly increased robustness with respect to disturbance variables for a configuration of the control of the volume flow control device because the dynamic requirements for the control of the valve can be reduced during the exhalation phase compared to the dynamic requirements for the control of the valve during the inhalation due to the structural configuration of the valve.

It may also occur in alternative implementations that the valves are actuated in exactly the opposite manner to achieve such an effect and they are closed at the beginning of a breathing phase. For example. the volume flow control device may then have a greater inertia (lower attenuation/lower dampening) during opening than during closing. Different circuit variants, especially parallel connections, series connections, bypass connections, which can be correspondingly implemented with valve assemblies with different attenuation focal points (during opening or during closing), are conceivable in exemplary embodiments.

The volume flow control device may have in some exemplary embodiments a pneumatic control element for controlling the volume flow by means of a control pressure volume, in which case a limitation of a control pressure volume change rate during opening differs from a limitation of the control pressure volume change rate occurring during closing.

The pneumatic control/regulation may offer, on the whole, a lower inertia compared, for example, to electromagnetic controls. For example, the pneumatic control element may comprise a pilot valve (which can be actuated pneumatically/electrically), which can be adjusted by means of a control pressure volume, or a pneumatic pump (which can be actuated electrically).

The volume flow control device may have a diaphragm valve, which can be controlled by means of the pneumatic control element. As a result, the volume flow can be set and controlled efficiently. The diaphragm valve may be able to be actuated via an admission connection and via a relief connection, wherein the admission connection and the relief connection may have different restrictions as limitations. The different restrictions represent an efficient action for achieving the different attenuations.

The different restrictions may be able to be adjusted, as a result of which an additional adaptation to the respective conditions, e.g., to the patient, can be made possible. The volume flow control device may further comprise a control device for the dynamic control of the restrictions, so that these can also be adapted in the course of a ventilation.

The admission connection and the relief connection may further have a common restriction in some exemplary embodiments. A basic attenuation can thus be defined for both directions.

The pneumatic control element may comprise a pneumatic pump, which can be actuated, for example, electrically. The volume flow control device may have a diaphragm valve, which can be controlled by means of the pneumatic control element, and which can be actuated via a control connection. The volume flow control device may comprise an electric control element for actuating the pneumatic pump. The pneumatic pump can thus be efficiently integrated into a regulating or control circuit and act as a final control element.

The valve assembly may be configured such that a restriction, which is configured, for example, for the definition of the fundamental attenuation, is arranged in the control connection. The restriction in the control connection may likewise be adjustable and thus be adapted to the respective conditions.

Exemplary embodiments create, moreover, a ventilator with a valve assembly described here for carrying out an inhalation.

Exemplary embodiments create, moreover, a ventilator with a valve assembly described here for carrying out an exhalation.

The volume flow control devices for the inhalation and for the exhalation may have a lower attenuation during opening than during closing. As a result, the volume flow changes can be changed at the beginning of the breathing phases at the desired rate, and an attenuation of disturbance variables does not have to be abandoned with a comparable volume change rate.

At least one of the volume flow control devices for the inhalation and for the exhalation may be configured to allow, relative to the same unit of time, volume flow changes during the opening that are higher than the volume flow changes occurring during the closing at least by a factor of 2, 4 or 8. An appropriate ratio of the rate change to the attenuation can be selected hereby.

At least one of the volume flow control devices for the inhalation and for the exhalation may be configured to allow, relative to the same unit of time, patient pressure changes during the opening that exceed the patient pressure changes occurring during closing at least by a factor of 2, 4 or 8. An appropriate ratio of rate change to attenuation can be selected by means of the patient pressure change as well.

Another exemplary embodiment is a ventilation system with a ventilator being described here.

Exemplary embodiments also create a process of operating a valve assembly in a ventilator. The valve assembly comprises an inlet for the inflow of a ventilation gas, an outlet for the outflow of the ventilation gas and a volume flow control device for the ventilation gas between the inlet and the outlet. The process comprises a setting of the volume flow of the ventilation gas in a range between shut-off and a maximum flow. The valve assembly is opened here with a first attenuation, wherein the volume flow of the ventilation gas increases, and the valve assembly is closed with a second attenuation, wherein the volume flow of the ventilation gas decreases. The first attenuation differs from the second attenuation.

Another exemplary embodiment is a computer program with a program code for carrying out one of the processes being described here when the program code is executed on a computer, on a processor or on a programmable hardware component.

Some examples of devices and/or processes will be explained in more detail below with reference to the attached figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view showing an exemplary embodiment of a valve assembly and an exemplary embodiment of a ventilator;

FIG. 2 is a block diagram of an exemplary embodiment of a process for operating a valve assembly in a ventilator;

FIG. 3 is a schematic view showing an exemplary embodiment of a ventilation system with typical components;

FIG. 4 is a schematic view showing an exemplary embodiment of a ventilation system with pneumatic pilot valves;

FIG. 5 is a graph showing a view of a typical ventilation curve (pressure time relationship) with the breathing phases;

FIG. 6 is a view of a breathing system with pilot valves and with specific attenuation in an exemplary embodiment;

FIG. 7 shows a view of different frequency responses as a function of Bode diagrams with low-pass character in exemplary embodiments;

FIG. 8 is a schematic view showing another exemplary embodiment;

FIG. 9 shows a Bode diagram for the exemplary embodiment according to FIG. 8;

FIG. 10 is a schematic view showing another exemplary embodiment;

FIG. 11 shows a Bode diagram for the exemplary embodiment from FIG. 10;

FIG. 12 is a schematic view showing another exemplary embodiment with simple attenuation;

FIG. 13 is a schematic view showing an exemplary embodiment with separate attenuation for load/relief; and

FIG. 14 is a schematic view showing an exemplary embodiment with adjustable attenuation.

### DESCRIPTION OF PREFERRED EMBODIMENTS

Referring to the drawings, different examples will now be described in more detail with reference to the attached figures. The thicknesses of lines, layers and/or areas may be exaggerated for illustration in the figures.

Further examples may cover modifications, equivalents and alternatives, which fall within the scope of the disclosure. Identical or similar reference numbers pertain in the entire description of the figures to identical or similar elements, which may be implemented identically or in a modified form in a comparison with one another, while they provide the same function or a similar function.

It is apparent that if an element is described as being “connected” or “coupled” with another element, the elements may be connected or coupled directly or via one or more intermediate elements. If two elements A and B are combined with the use of an “or,” this shall be defined such that all possible combinations are disclosed, i.e., only A, only B as well as A and B, unless something else is explicitly or implicitly defined. An alternative wording for the same combinations is “at least one of A and B” or “A and/or B.” The same applies, mutatis mutandis, to combinations of more than two elements.

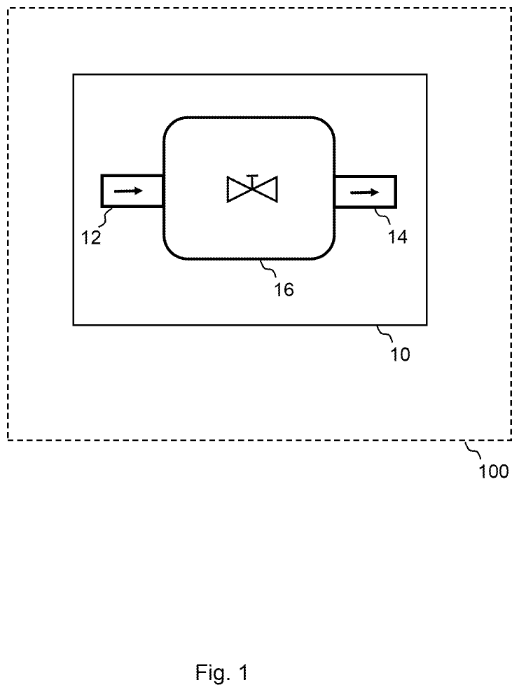

FIG. 1 shows an exemplary embodiment of a valve assembly 10 and an exemplary embodiment of a ventilator 100.

The valve assembly 10 for a ventilator 100 (shown by broken lines, because it is optional from the viewpoint of the valve assembly) comprises an inlet 12, which is configured for the inflow of a ventilation gas. The valve assembly 10 further comprises an outlet 14, which is configured for the outflow of the ventilation gas. Moreover, the valve assembly 10 comprises a volume flow control device 16 for the ventilation gas between the inlet 12 and the outlet 14. The volume flow control device 16 is configured to set the volume flow of the ventilation gas in a range between shut-off and a maximum volume flow. The volume flow control device 16 is configured such that an attenuation of a volume flow change occurring during the opening, when the volume flow of the ventilation gas is increased, differs from an attenuation occurring during the closing, when the volume flow of the ventilation gas is reduced.

As is shown as an option (drawn in broken lines) in FIG. 1, a ventilator 100 (or ventilation system 100) with a valve assembly 10 for inhalation and/or with a valve assembly 10 for exhalation is another exemplary embodiment.

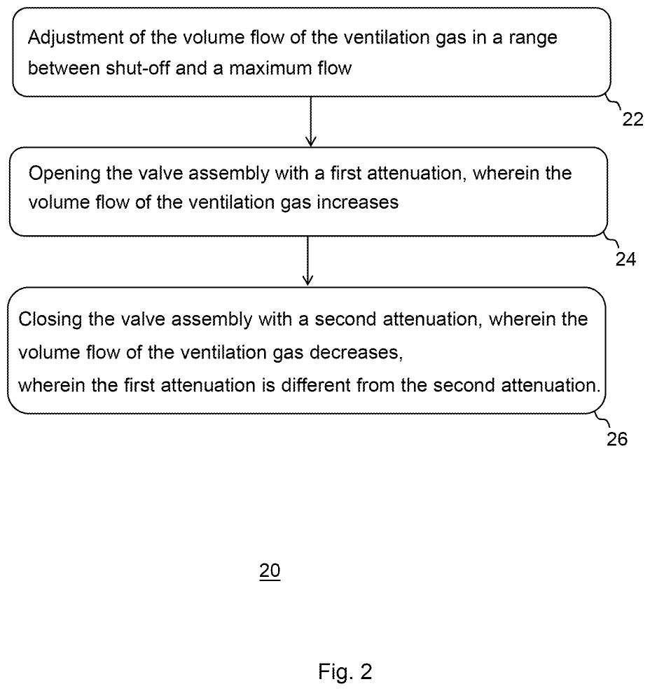

FIG. 2 shows a block diagram of an exemplary embodiment of a process 20 for operating a valve assembly 10 in a ventilator 100. The process 20 for operating a valve assembly 10 in a ventilator 100 comprises the setting 22 of the volume flow of the ventilation gas in a range between shut-off and a maximum flow. The process 20 further comprises an opening 24 of the valve assembly 10 with a first attenuation, when the volume flow of the ventilation gas increases, and a closing 26 of the valve assembly 10 with a second attenuation, when the volume flow of the ventilation gas decreases. The first attenuation differs from the second attenuation.

In some exemplary embodiments, an adaptation of a pneumatic control system can be carried out by an attenuator. This attenuator extracts energy from the system as soon as the output variable changes rapidly. Such an excited deflection of the output variable cannot thus lead so easily to a permanent or even rising oscillation amplitude any more. However, this attenuator comes into action mainly in case of the undesired excitations (disturbance variables) in order not to influence the actual ventilation performance in the sense of a rapid pressure increase or in order not to influence it too strongly.

Digital controllers are, in principle, also able to amplify known frequencies of a control system selectively more weakly and hence to reduce or to suppress an oscillation. However, this requires a corresponding computing capacity.

Some exemplary embodiments use an attenuation set at a fixed value, which is integrated into a control system. As a result, the controller is relieved and more effort can correspondingly be expended for the reduction of specifically occurring resonance step-ups.

Thus, the volume flow control device 16 may have a lower attenuation during the opening than during closing or also vice versa, depending on the direction in which a higher dynamics (faster change) is desired. This dynamics may vary depending on the circuitry and the particular application; as will be explained in more detail below, a faster change is desired during ventilation at the beginning of a breathing phase. Such a fast change can be brought about in terms of the circuitry by a fast opening or also by a fast closing. The volume flow control device 16 can therefore also have a higher inertia during opening than during closing, depending on the particular variant of use and circuitry.

Some exemplary embodiments, in which the volume flow control device 16 has a pneumatic control element for controlling the volume flow on the basis of a control pressure volume, will be explained in more detail below. A limitation of a control pressure volume change rate during the opening differs from a limitation of the control pressure volume change rate occurring during closing.

This can be achieved by a correspondingly integrated attenuation. The attenuation is integrated in this case specifically so that the intended, fast responses of the system will not be limited or they will only be limited to a lesser extent and the greatest possible attenuation acts on the unintended, fast disturbance variables.

For example, the attenuation element shall extract energy from the system as soon as an oscillation generates an excessively steep slope of a signal.

It is useful for this to present the motion equation for a mechanical, oscillating model, which will be done below—as is also explained on the basis of Formula 1—based on the example of a diaphragm valve.

E=M*\ddot{s}(t)+D*\dot{s}(t)+F*s(t) Formula 1

in which E is the total force,
M is the mass,
D is the attenuation or friction with a corresponding attenuation constant and F is a spring with a corresponding spring rate.
s(t) is the system in the time-related representation, i.e., e.g., the motion of a diaphragm;
\dot{s}(t) is the first derivative according to time, i.e., the velocity of the diaphragm, and
\ddot{s}(t) is the second derivative according to the time, i.e., an acceleration of the diaphragm.

The mass is the mass of the diaphragm and of all moving parts in this example.

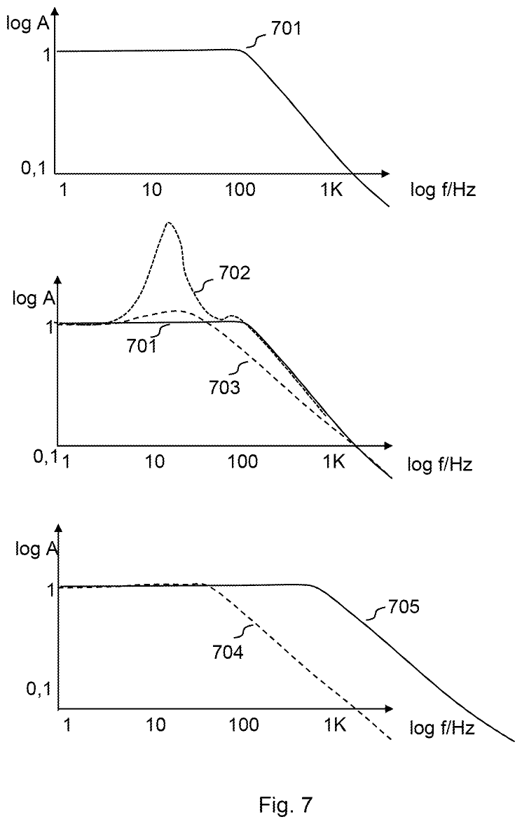

The attenuation is represented here, for example, by the viscosity of a diaphragm suspension or also by other forces, which are generated when the diaphragm shall change at a velocity. The attenuation can be used, in particular, to effectively suppress a resonance step-up by the combination of a plurality of components that are able to oscillate, as this will also be explained in more detail below on the basis of FIG. 7.

The spring sets the path-dependent force. This is, as a rule, the actuating variable, with which the position of the diaphragm is set. The system itself also contains a spring characteristic, which is combined with the actuating variable.

Many components, which each generate a special behavior of their own in response to an excitation or also generate excitations themselves, are present in a pneumatic system, as it is given, for example, in a ventilation system.

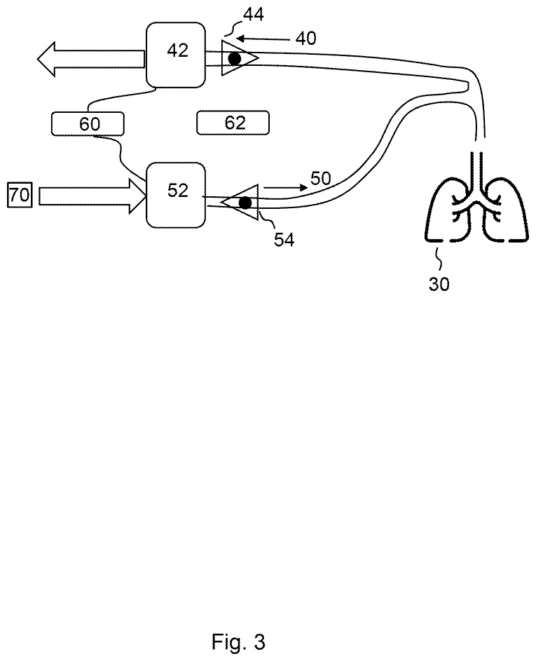

FIG. 3 shows an exemplary embodiment of a ventilation system with typical components. The right-hand side of FIG. 3 shows a patient, who is symbolized by a schematically shown lung 30. This lung is coupled via a Y connection by means of ventilation tubes to an exhalation path 40 and to an inhalation path 50. An exhalation valve 42 and a nonreturn valve 44, which prevents a volume flow towards the patient, are located in the exhalation path 40. An inhalation valve 52 and a nonreturn valve 54, which prevents a volume flow away from the patient, are analogously located in the exhalation path 50. The exhalation valve 42 and the inhalation valve 52 are coupled with sensors 60, which detect pressures or volume flows. The system may also comprise at different locations additional sensors 62, which detect corresponding measured variables for the control. The inhalation valve 52 is coupled on the inlet side to a gas source 70 for providing the breathing gas. These components are also present in the exemplary embodiments explained below and will not be described again.

The excitations occurring during the normal operation are the changes in the desired variables due to the ventilation control, e.g., at the time of the change between the ventilation phases from inhalation to exhalation and vice versa. This excitation shall be transmitted to the system as rapidly as possible and in the asymptotic borderline case to the system. Other excitations of other effects or components of the system shall be attenuated to the extent possible. This means that the sum of the responses from all components must be lower than the amplification 1.

In the ideal case this means that, for example, an excitation caused by a mechanical bumping onto a ventilation tube does not generate a pressure oscillation. Through a poorly attenuated system, such an excitation (pressure velocity wave) can propagate to all components in the system with low losses. If the response of the other components is at a value of 1 (the impedance jump is correspondingly reverberant) or even higher, the excitation at the next oscillation amplitude is at least just as high as or even higher than the first excitation. The system then progresses to an oscillation.

Furthermore, each component of the system is provided with a frequency response of its own. This can be represented as a Bode diagram. Since the oscillation is an energy and mass exchange, there also are strongly nonlinear frequency curves. Resonance ranges develop as a function of the frequency. The frequencies of resonance, qualities (width of the resonance range) and amplitudes are only partially constant. Some components are, e.g., strongly temperature-dependent (diaphragms made of elastomers) or even individual and variable (patient compliance and resistance are variable over time).

The exceeding of the frequency of resonance likewise ensures a phase change, as a result of which the response of the system will be shifted by, for example, 180°, and an imaginary negative feedback of a controller becomes a positive feedback for disturbance variables.

This could be counteracted by the attempt at integrating in the system so many components with a low-pass function that a rapid response will be rapidly suppressed. This unfortunately leads to slow overall behavior of the system, as a result of which the pressure rise time would not be sufficient for a ventilation.

At least some exemplary embodiments generate a high attenuation by a restriction, i.e., by a resistance or by a narrowing in a pneumatic system. The carrying through of a volume flow through this restriction generates a high pressure loss, which also leads as a result to a loss of energy.

On a closer scrutiny of the components, the beginning of the breathing phases is always the time range in which high pressure gradients are required and desirable. For the inhalation valve 42, this is the beginning of the inhalation. An attenuated characteristic is rather desired for the rest of the course of the ventilation. As a result, even though disturbance variables can be compensated more slowly, no excessive system response amplitudes will develop any longer.

A control system for a pneumatic pressure/volume flow control can be configured in the ventilation technology in exemplary embodiments such that an operation in a broad frequency range is made possible at a reasonable effort for the controller. Suppression of oscillation excitations can in this case be suppressed with passive elements. This leads to a lower load on the computing capacity, for example, a software controller. A fundamental attenuation can thus be achieved in exemplary embodiments by pneumatic elements, and tuning can be carried out by parameterization of the controller.

The pneumatic control element is in some exemplary embodiments, for example, a pilot valve, which can be set by means of a control pressure volume, or a pneumatic pump.

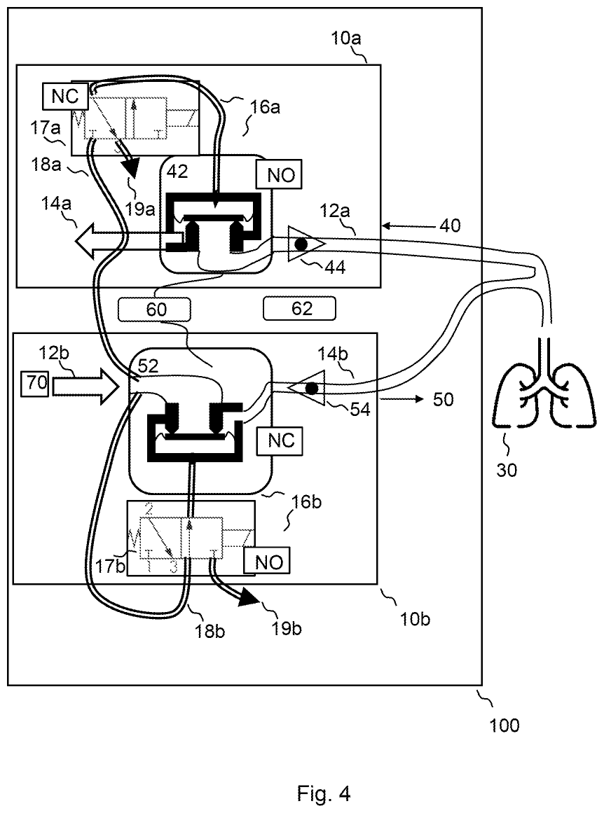

FIG. 4 shows an exemplary embodiment of a ventilation system or of a ventilator 100 with pneumatic pilot valves 17a, 17b. The ventilator 100 comprises, based on the components shown in FIG. 3, a valve assembly 10a and 10b each in the exhalation path 40 and in the inhalation path 50. The valve assembly 10a comprises an inlet 12, an outlet 14a and a volume flow control device 16a. The valve assembly 10b analogously comprises an inlet 12b, an outlet 14b and a volume flow control device 16b. The volume flow control devices 16a and 16b have a diaphragm valve 42, 52 (exhalation valve 42, inhalation valve 52) each, which can be controlled by means of the pneumatic control element (pilot valves 17a, 17b), wherein the diaphragm valve 42, 52 can be actuated via a respective admission connection 18a, 18b and via a respective relief connection 19a, 19b (discharge into the atmosphere). Different implementations of the valves concerning the normal states thereof (currentless resting state) are conceivable in different exemplary embodiments. For example, distinction can thus be made between normally open (NO) and normally closed (NC) valves. The pilot valve 17a in the exhalation path 40 is implemented as an NC and the exhalation valve 42 as an NO in the exemplary embodiment shown in FIG. 4. The pilot valve 17b can be an NO and the inhalation valve 52 can be an NC in the inhalation path 50. Other implementations, especially reversed implementations, are also conceivable in other exemplary embodiments. These components also occur in the exemplary embodiments explained below and will not be described again.

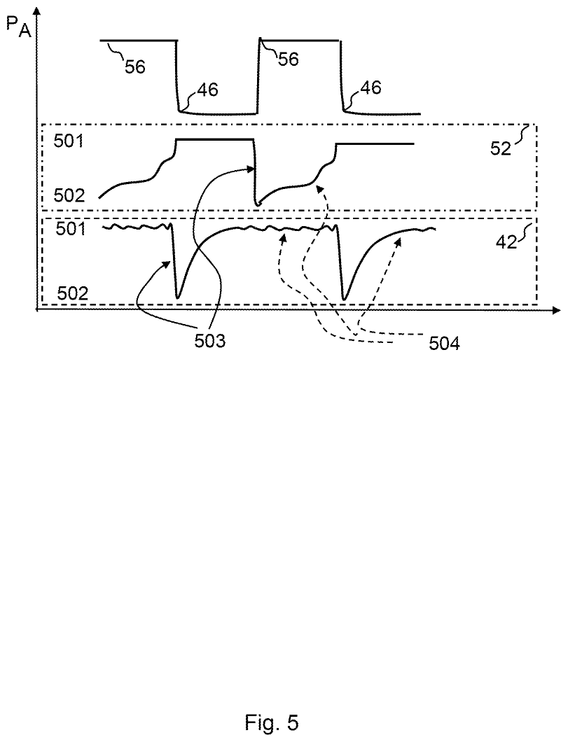

FIG. 5 shows a view of a typical ventilation curve with the breathing phases. FIG. 5 shows a time curve diagram, in which the time is plotted to the right and the pressure PAW and, also qualitatively, the position of the diaphragm is plotted upwards. The schematic curve of the breathing phases with the inhalation phase 56 and with the exhalation phase 46 is shown at the top. The pressure is qualitatively high during the inhalation phase 56 in order to bring about a volume flow in the direction of the patient during the inhalation and the pressure is low during the exhalation phase 46 in order to bring about a volume flow away from the patient during the exhalation. FIG. 5 shows in its central part the curve of the diaphragm setting or position of the inhalation valve 52 and the curve of the diaphragm setting or position of the exhalation valve 42 at the bottom. The valve is always closed in the curves shown at the top, 501, and it is correspondingly opened at the bottom 502.

As can be seen in FIG. 5, the inhalation valve 52 opens at the beginning of the inhalation phase 56 abruptly (rapidly) and then closes again slowly over the course of the inhalation phase 56. The exhalation valve 42 analogously opens at the beginning of the exhalation phase 46 abruptly (rapidly) and then closes slowly over the course of the exhalation phase 46. The rapid opening of the valves 42, 52 is highlighted by the arrows 503 in FIG. 5. As is also seen in FIG. 5, control operations, which are manifested in smaller fluctuations of the diaphragm positions (dynamic control) and which are highlighted by the arrows 504 in FIG. 5, take place during the closing process of the valves 42, 52 in the course of the phases 46, 56.

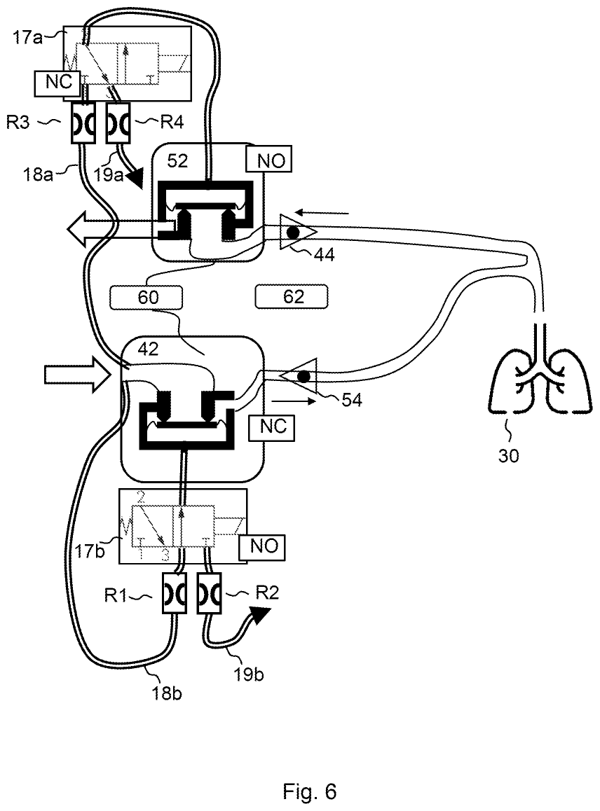

FIG. 6 shows a representation of a ventilation system with pilot valves 17a, 17b and with specific attenuation in an exemplary embodiment. FIG. 6 shows the device from FIG. 4 with the same components, the admission connections 18a, 18b and the relief connections 19a, 19b having different restrictions R1, R2, R3, R4 as limitations. For example, 40 mbar/(L/minute) is always selected for the restrictions R1 and R3 in the admission connections 18a, 18b and 5 mbar/(L/minute) is always selected for the restrictions R2 and R3 in the admission connections 19a, 19b.

Accordingly, a restriction R1, R2, R3, R4 is integrated in some exemplary embodiments into the actuation of a valve with the pilot valves, separately for inhalation and for exhalation on the sides 18a, 18b, which are not necessary for the rapid pressure change. The attenuation shall in this case especially prevent the transmission of a disturbance variable as a positive feedback to the control system. An attenuation in the ventilation system itself is unaffected thereby. This has the advantage that no additional active elements are present in the system.

As was already stated, an attenuation is integrated in this case for disturbance variables. The intended fast changes at the beginning of a breathing phase 46, 56 shall not be attenuated if possible or they should be attenuated to a lesser extent only.

In the case of the inhalation valve 42, the valve is configured as an NC (NORMALLY CLOSED) type valve. This embodiment was already shown schematically in FIG. 4. The pilot valve 17b is accordingly an NO (NORMALLY OPEN) type valve in order to keep the inhalation valve 42 closed. At the beginning of the inhalation 46, the inhalation valve 42 shall be able to open as fast as possible and to open as wide as possible. During the other time of the inhalation 46, only the compliance (here especially of the patient) and possibly disturbance variables shall be compensated. The inhalation valve 42 remains almost completely closed during the exhalation 56 and it compensates only leaks and possibly disturbance variables. Such a valve assembly including the restrictions R1, R2, R3, R4 is shown schematically in FIG. 6. The restrictions are used to attenuate the system and are configured for the relief with a low resistance value R2. The admission of a higher pressure takes place through a restriction with a higher resistance value. As a result, an attenuation develops, which acts predominantly on the closing of the inhalation valve 42 but generates no attenuation or a slight attenuation for the opening.

As was explained farther above, it holds true for disturbance variables and oscillation excitations that energy shall be extracted from the system, and the distribution of the attenuation between opening/closing is irrelevant for the overall attenuation. Similar requirements arise for the exhalation valve 52. The exhalation valve 52 has the NO (NORMALLY OPEN) type configuration here, so that the pilot valve 17a has the NC (NORMALLY CLOSED) type configuration. The venting of the exhalation valve 52 must take place rapidly during the initiation of the exhalation 56, whereas the closing can take place in an attenuated manner. The restriction R4 for the relief is small and the restriction R3 for the admission is higher here as well.

The above-mentioned NC and NO valve types differ according to the usual technical teaching as follows:
An NO valve is in the “OPEN” state without external activation, i.e., a gas can flow through the valve.
An NC valve is in the “CLOSED” state without external activation, i.e., no gas can flow through the valve.

For example, the following values are selected as values for the restriction in case of a control pressure volume of 5 mL:

Inhalation:
Admission R1=40 mbar/(L/minute) Relief R2=5 mbar/(L/minute)

Exhalation:
Admission R3=40 mbar/L/minute) Relief R4=5 mbar/(L/minute).

FIG. 7 shows a view of some frequency responses with Bode diagrams and low-pass character in exemplary embodiments. FIG. 7 shows Bode diagrams, which show the logarithmically plotted amplitude log A upwards compared to the frequency in Hz log f/Hz, which is plotted logarithmically to the right. FIG. 7 shows in its top part a classical low-pass curve 701, in which the attenuation increases steadily starting from a limit frequency and the amplitude drops correspondingly. FIG. 7 shows in its central part, by contrast, a curve 702 with a resonance, i.e., an amplitude step-up (amplification) without attenuation, and a curve 703 with a resonance with adapted attenuation in one exemplary embodiment. FIG. 7 shows, moreover, in its bottom part a comparison between a frequency response 704 with strong attenuation (lower limit frequency) and a frequency response 705 with lower attenuation (higher limit frequency).

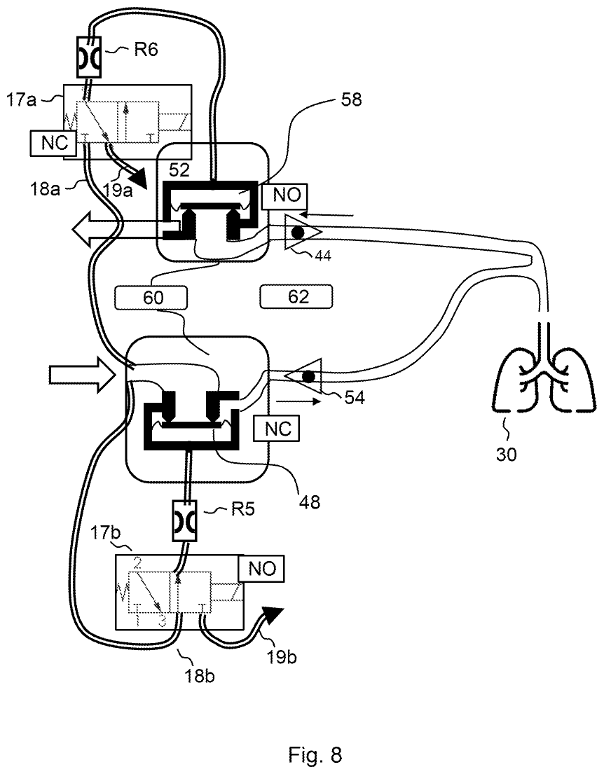

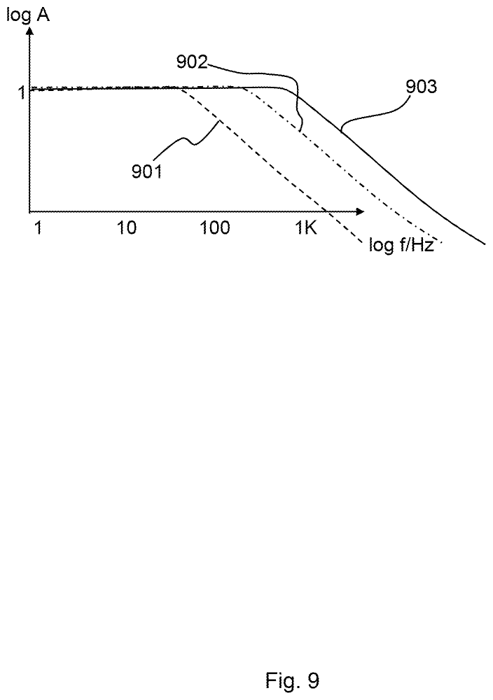

FIG. 8 shows another exemplary embodiment, in which a restriction R5, R6 each was introduced into the control lines/control connection. FIG. 9 shows a Bode diagram for the exemplary embodiment from FIG. 8.

This exemplary embodiment pertains to the introduction of additional energy extraction systems, which can or must each be set. The embodiment is shown for this with restrictions and with a volume located behind the restriction. The two valves 42, 52 to be controlled have control pressure volumes 48, 58, which are located each behind the diaphragms.

The limit frequency can be set in this case according to Formula 2 with the value of the volume (C for volume capacity) and with the restriction R5, R6:

ω=1/(2π*R*C) Formula 2.

For example, the restrictions R5, R6=10 mbar/(L/minute) are selected. The reduction of the limit frequency is also helpful in suppressing the transmission of higher frequency components. The maintenance of the volume may be especially advantageous in this case in adapting the restriction, because especially the energy extraction can be modulated here. The volume of the inhalation valve 48 and of the exhalation valve 58 for the control pressure can be used here for the volume (see FIG. 8). FIG. 9 shows the frequency response with strong attenuation 901 (e.g., R1, R3 according to the above description), with moderate attenuation 902 (e.g., R5, R6 according to the above description) and with low attenuation 903 (e.g., R2, R4 according to the above description).

In another exemplary embodiment, one or more restrictions can be set permanently, variably or dynamically, for example, also by a control device. As a result, these elements can be set dynamically in order nevertheless to make possible a sufficiently fast system response in the case of an intended pressure change in case of sufficient disturbance variable suppression.

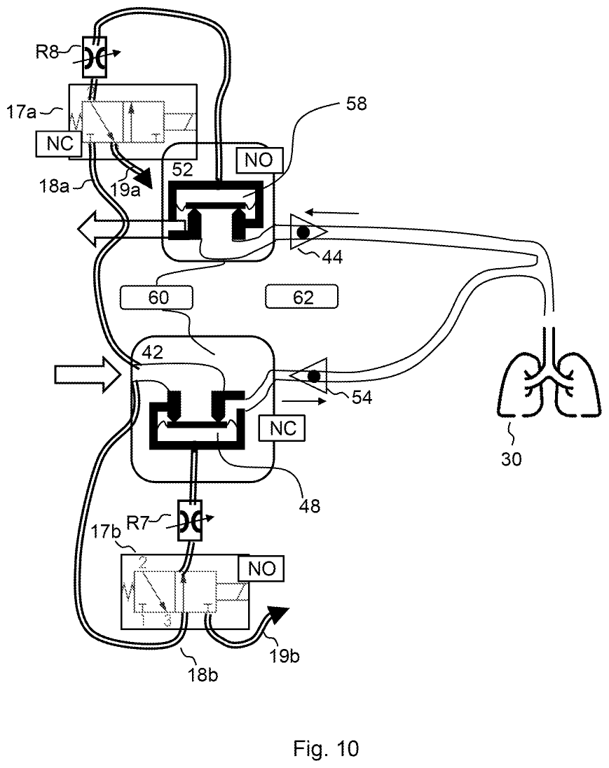

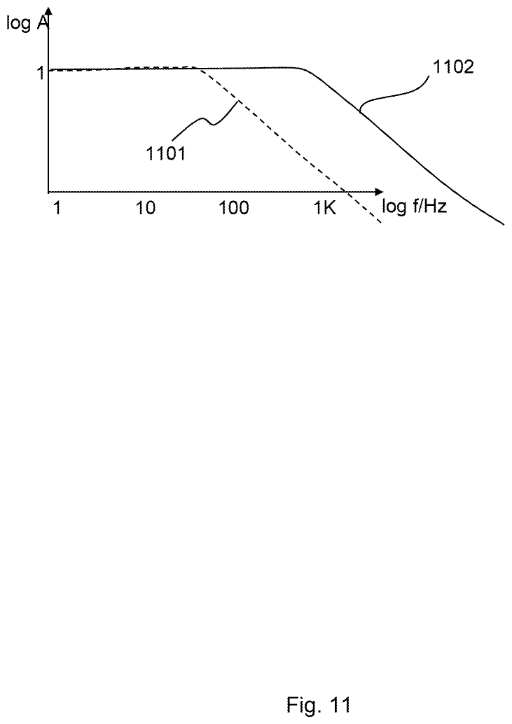

FIG. 10 shows another exemplary embodiment, in which the attenuation can be set in the control line/control connection. FIG. 11 shows a Bode diagram for the exemplary embodiment from FIG. 10. The restrictions R7, R8 can be set in these exemplary embodiments, e.g., in a range of 1 . . . 40 mbar/(L/minute). The volume flow control device 16 can then further comprise a control device for the dynamic control of the restrictions R7, R8. The admission connection and the relief connection may further also have a common restriction. FIG. 11 shows on the basis of the corresponding Bode diagram the frequency response with strong attenuation 1101 (high R7 and R8) as well as with weak attenuation 1102 (low R7 and R8).

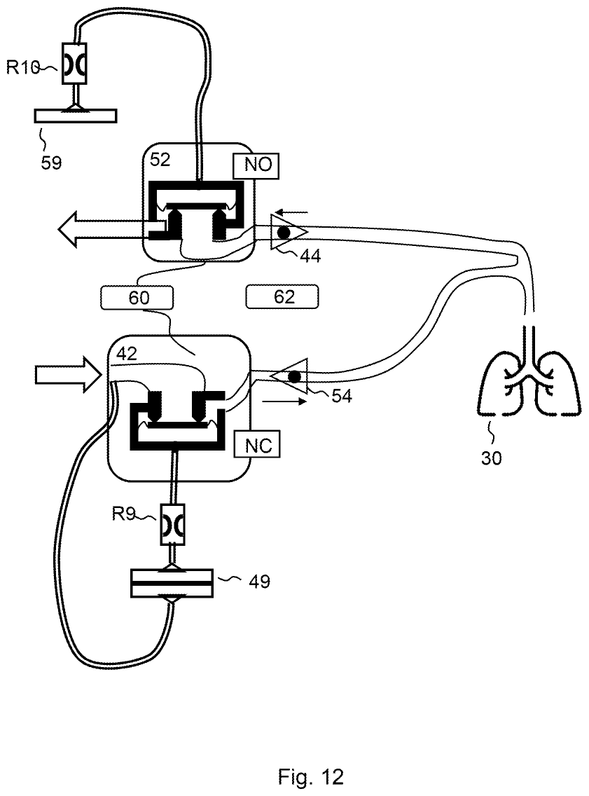

In other exemplary embodiments, the pneumatic control element may comprise a pneumatic pump 49, 59. FIG. 12 shows another exemplary embodiment with simple attenuation R10, similarly to the exemplary embodiment shown in FIG. 8. As is shown in FIG. 12, the restrictions R9, R10, which are dimensioned, for example, with 10 mbar/(L/minute), are located each in the control connections between the pump 49, 50 and the valve 42, 52. The volume flow control device comprises in this exemplary embodiment a diaphragm valve 42, 52 each, which is controllable by means of the pneumatic control element 49, 59. The diaphragm valve 42, 52 can be actuated via a control connection. The volume flow control device may comprise, moreover, an electrical control element for actuating the pneumatic pump, for example, a controller, a processor or programmable hardware. In the same manner, adjustable restrictions may also be controlled or regulated electronically in other exemplary embodiments. Another exemplary embodiment is therefore also a computer program with a program code for carrying out one of the processes being described here when the program code is executed on a computer, on a processor or on a programmable hardware component.

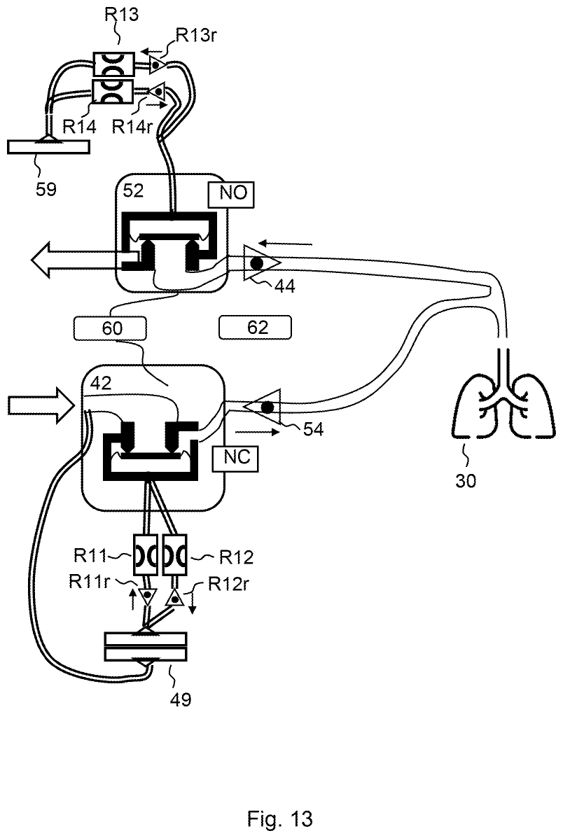

FIG. 13 shows an exemplary embodiment with respective separate attenuation for the load R11, R14 (e.g., 5 mbar/(L/minute) and relief R12, R13 (e.g., 40 mbar/(L/minute). An (anti)-parallel circuit comprising a restriction (R11, R12, R13, R14) and a nonreturn valve (R11r, R12r, R13r, R14r) each is arranged for this purpose downstream of the pneumatic pumps 49, 59 in the control connection. The nonreturn valves (R11r, R12r, R13r, R14r) ensure that the breathing gas can flow in one direction only in the respective branch. As a result, different attenuations can be achieved during opening and during closing. Thus, the inhalation valve 42 can only be opened via the restriction R12 and via the nonreturn valve R12r in FIG. 13, and the exhalation valve 52 analogously only via R13 and the nonreturn valve R13r. In the same manner, the inhalation valve 42 can only be closed via the restriction R11 and the exhalation valve 52 can only be closed via R14. The opening and closing dynamics can therefore be influenced by an appropriate selection of the restrictions.

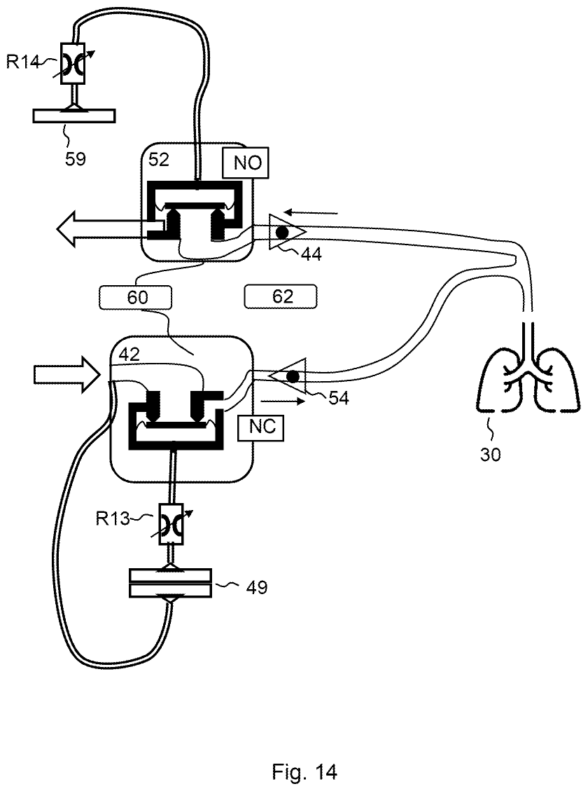

Analogously to the above-described exemplary embodiments, adjustable attenuations can also be used in exemplary embodiment with micropumps 49, 59. FIG. 14 shows an exemplary embodiment with adjustable attenuation. Analogously to the above exemplary embodiments, the attenuations may likewise be built for load and relief separately in the embodiments with pump 49, 59 (see FIG. 13) and/or they may also be adjustable, as this is shown in FIG. 14. The two restrictions R13, R14 in the control connections between the pump 49, 59 and the valve 42, 52 have an adjustable configuration here, for example, in a range of 1 . . . 40 mbar/(L/minute).

The volume flow control devices for the inhalation and for the exhalation are configured in the exemplary embodiments being explained here such that there is a lower attenuation during opening than during closing. Other exemplary embodiments or implementations are generally conceivable as well.

For example, at least one of the volume flow control devices is configured for the inhalation and for the exhalation in order to allow, relative to the same unit, volume flow changes during opening that exceed the volume flow changes occurring during closing at least by a factor of 2, 4 or 8. In a concrete implementation, the volume flow change can be limited to 100 L/minute in 30 msec during the increase and to 100 L/minute in 240 msec during lowering.

At least one of the volume flow control devices for the inhalation and for the exhalation may be configured to allow, relative to the same unit of time, patient pressure changes during opening that exceed the patient pressure changes occurring during closing at least by a factor of 2, 4 or 8. The patient pressure change can thus be limited in one implementation to 40 mbar in 30 msec during the increase and to 40 mbar in 240 msec during the lowering.

The aspects and features that are described together with one or more of the examples and figures described in detail above may also be combined with one or more of the other examples in order to replace an identical feature of the other example or in order to additionally introduce the feature into the other example.

Examples may be or pertain to, furthermore, a computer program with a program code for executing one or more of the above processes when the computer program is executed on a computer or processor. Steps, operations or processes of different processes described above may be executed by programmed computers or processors. Examples may also cover program storage devices that are tangible and non-transient, e.g., digital storage media, which are machine-readable, processor-readable or computer-readable and machine-executable, processor-executable or computer-executable programs of instructions. The instructions execute some or all of the steps of the above-described processes or cause them to be executed. The program storage devices may comprise or be, e.g., digital memories, magnetic storage media, for example, magnetic disks and magnetic tapes, hard drives or optically readable digital storage media. Further examples may also cover computers, processors or control units, which are programmed for executing the steps of the above-described processes, or (field)-programmable logic arrays ((F)PLAs=(Field) Programmable Logic Arrays) or (field)-programmable gate arrays ((F)PGA=(Field) Programmable Gate Arrays), which are programmed for executing the steps of the above-described processes.

Only the basic principles of the disclosure are shown by the description and the drawings. Furthermore, all the examples listed here shall be used, in principle, expressly for illustrative purposes only in order to support the reader in understanding the basic principles of the disclosure and of the concepts contributed by the inventor(s) to the further development of the technique. All the statements made here about basic principles, aspects and examples of the disclosure as well as concrete examples thereof comprise the equivalents thereof.

A function block designated as a “means for . . . ” carrying out a defined function may pertain to a circuit, which is configured for carrying out a defined function. A “means for something” can thus be implemented as a “means configured for or suitable for something,” e.g., configured as a component or a circuit for or suitable for the respective task.

Functions of different elements shown in the figures including each function block designated as “means,” “means for providing a signal,” “means for generating a signal,” etc., may be implemented in the form of dedicated hardware, e.g., “a signal provider,” “a signal processing unit,” “a processor,” “a control,” etc., as well as as hardware capable of executing software in connection with corresponding software. In case of provision by a processor, the function may be provided by an individual, jointly used processor or by a plurality of individual processors, some of which or all of which can be used jointly. However, the term “processor” or “control” is far from being limited to hardware capable exclusively of executing software, but it may comprise digital processor hardware (DSP hardware, DSP=Digital Signal Processor), network processor, application-specific integrated circuit (ASIC=Application Specific Integrated Circuit), field-programmable logic array (FPGA=Field Programmable Gate Array), read-only memory (ROM=Read Only Memory) for storing software, random-access memory (RAM=Random Access Memory) and non-volatile storage device (storage). Other hardware, conventional and/or customer-specific, may also be included.

A block diagram may represent, for example, a schematic circuit diagram, which implements the basic principles of the disclosure. Similarly, a flow chart, a flow diagram, a state transition diagram, a pseudocode and the like may represent different processes, operations or steps, which are represented, for example, essentially in computer-readable medium and are thus executed by a computer or by a processor, regardless of whether such a computer or processor is explicitly shown. Processes disclosed in the description or in the patent claims may be implemented by a component that has means for carrying out each of the respective steps of this process.

It is apparent that the disclosure of a plurality of steps, processes, operations or functions disclosed in the description shall not be interpreted as being in the defined order, unless this is explicitly or implicitly stated otherwise, e.g., for technical reasons. Therefore, these are not limited to a defined order by the disclosure of a plurality of steps or functions, unless these steps or functions are not replaceable for technical reasons. Further, an individual step, function, process or operation may include in some examples a plurality of partial steps, partial functions, partial processes or partial operations and/or may be broken up into same. Such partial steps may be included and be part of the disclosure of these individual steps, unless they are explicitly ruled out.

Furthermore, the following claims are herewith included in the detailed description, where each claim may stand as a separate example in itself. While each claim may stand in itself as a separate example, it should be borne in mind that—even though a dependent claim may relate in the claims to a defined combination with one or more other claims, other examples may also comprise a combination of the dependent claim with the subject of every other dependent or independent claim with the subject of every other dependent or independent claim. Such combinations are explicitly proposed here, unless it is stated otherwise that a defined combination is not intended. Further, features of a claim may also be included for every other independent claim, even if this claim is not made directly dependent on the independent claim.

While specific embodiments of the invention have been shown and described in detail to illustrate the application of the principles of the invention, it will be understood that the invention may be embodied otherwise without departing from such principles.

### LIST OF REFERENCE CHARACTERS

10, 10a, 10b Valve assembly
12, 12a, 12b Inlet
14, 14a, 14b Outlet
16, 16a, 16b Volume regulating device
17a, 17b Pilot valves
18a, 18b Admission connection
19a, 19b Relief connection
20 Process for operating a valve assembly
22 Adjustment of the volume flow of the ventilation gas in a range between shut-off and a maximum flow
24 Opening of the valve assembly with a first attenuation, wherein the volume flow of the ventilation gas increases
26 Closing of the valve assembly with a second attenuation, wherein the volume flow of the ventilation gas decreases, wherein the first attenuation is different from the second attenuation
30 Patient/lung
40 Exhalation path
42 Exhalation valve
44 Nonreturn valve
46 Exhalation phase
48 Control volume
49 Pump, micropump, pneumatic pump
50 Inhalation path
52 Inhalation valve
54 Nonreturn valve
56 Inhalation phase
58 Control volume
59 Pump, micropump, pneumatic pump
60 Sensors
62 Sensors
70 Gas source
100 Ventilator
501 closed
502 open
503 fast opening
504 dynamic control
701 Low-pass curve
702 Curve with resonance
703 Curve with a resonance and with adapted attenuation
704 greatly attenuated curve
705 slightly attenuated curve
901 greatly attenuated curve
902 moderately attenuated curve
903 weakly attenuated curve
1101 greatly attenuated curve
1102 weakly attenuated curve
R1-R14 Restrictions
R11r-R14r Nonreturn valves

What is claimed is:

1. A valve assembly for a ventilator, the valve assembly comprising:
an inlet configured for an inflow of a ventilation gas;
an outlet configured for an outflow of the ventilation gas; and
a volume flow control device configured to control the ventilation gas between the inlet and the outlet and further configured:
to set a volume flow of the ventilation gas in a range between shut-off and a maximum volume flow; and
to provide an attenuation of a volume flow change which volume flow change occurs during an opening, providing an increase in volume flow of the ventilation gas, that differs from an attenuation occurring during a closing, providing a reduction of the volume flow of the ventilation gas.

2. A valve assembly in accordance with claim 1, wherein the volume flow control device provides a lower attenuation of the volume flow change during opening than the attenuation of the volume flow change during closing.

3. A valve assembly in accordance with claim 1, wherein the volume flow control device has a higher attenuation of the volume flow change during closing than the attenuation of the volume flow change during opening.

4. A valve assembly in accordance with claim 1, wherein the volume flow control device has a higher inertia during opening than during closing.

5. A valve assembly in accordance with claim 1, wherein:
the volume flow control device comprises a pneumatic control element for controlling the volume flow by means of a control pressure volume; and
a limitation of a control pressure volume change rate, which limitation occurs during the opening, is different from a limitation of the control pressure volume change rate, which limitation occurs during the closing.

6. A valve assembly in accordance with claim 5, wherein the pneumatic control element comprises a pilot valve configured to be adjusted by a control pressure volume or a pneumatic pump.

7. A valve assembly in accordance with claim 5, wherein:
the volume flow control device comprises a diaphragm valve configured to be controlled by the pneumatic control element;
the diaphragm valve is configured to be actuated by an admission connection and by a relief connection; and
the admission connection and the relief connection each have limiting restrictions.

8. A valve assembly in accordance with claim 7, wherein the different restrictions are adjustable.

9. A valve assembly in accordance with claim 8, wherein the volume flow control device further comprises a control device for the dynamic control of the restrictions.

10. A valve assembly in accordance with claim 5, wherein:
the volume flow control device comprises a diaphragm valve configured to be controlled by the pneumatic control element;
the diaphragm valve is configured to be actuated by an admission connection and by a relief connection;
the admission connection and the relief connection have a common limiting restriction.

11. A valve assembly in accordance with claim 5, wherein:
the pneumatic control element comprises a pneumatic pump;
the volume flow control device further comprises a diaphragm valve configured to be controlled by the pneumatic control element;
the diaphragm valve is actuated via a control connection; and
the volume flow control device further comprises an electrical control element for actuating the pneumatic pump.

12. A valve assembly in accordance with claim 11, wherein a restriction is arranged in the control connection.

13. A valve assembly in accordance with claim 12, wherein the restriction in the control connection is adjustable.

14. A ventilator comprising:
a valve assembly comprising:
an inlet configured for an inflow of a ventilation gas;
an outlet configured for an outflow of the ventilation gas; and
a volume flow control device configured to control the ventilation gas between the inlet and the outlet and further configured:
to set a volume flow of the ventilation gas in a range between shut-off and a maximum volume flow; and
to provide an attenuation of a volume flow change which volume flow change occurs during an opening, providing an increase in volume flow of the ventilation gas that differs from an attenuation occurring during a closing, providing a reduction of the volume flow of the ventilation gas.

15. A ventilator in accordance with claim 14, wherein:
the ventilator is configured to carry out an inhalation phase; or
the ventilator is configured to carry out an exhalation phase; or
the ventilator is configured to carry out an inhalation phase and the ventilator is configured to carry out an exhalation phase.

16. A ventilator in accordance with claim 15, wherein:
the volume flow control device provides a lower attenuation for the inhalation; or
the volume flow control device provides a lower attenuation for the exhalation during the opening than during the closing; or
the volume flow control device provides a lower attenuation for the inhalation and the volume flow control device provides a lower attenuation for the exhalation during the opening than during the closing.

17. A ventilator in accordance with claim 16, wherein the volume flow control device is configured for the inhalation and for the exhalation to allow, relative to the same unit of time, volume flow changes during opening that exceed the volume flow changes occurring during closing at least by a factor of 2.

18. A ventilator in accordance with claim 16, wherein the volume flow control device is configured for inhalation and for the exhalation to allow, relative to the same unit of time, patient pressure changes occurring during opening that exceed patient pressure changes occurring during closing at least by a factor of 2.

19. A process for operating a valve assembly in a ventilator, wherein the valve assembly comprises an inlet for an inflow of a ventilation gas, an outlet for an outflow of the ventilation gas and a volume flow control device for control of the ventilation gas between the inlet and the outlet, the process comprising the steps of:
setting a volume flow of the ventilation gas in a range between shut-off and a maximum flow;
opening the valve assembly with a first attenuation, when the volume flow of the ventilation gas increases; and
closing the valve assembly with a second attenuation, when the volume flow of the ventilation gas decreases, wherein
the first attenuation differs from the second attenuation.

20. A process in accordance with claim 19, further comprising providing a computer program with a program code for carrying one for mores steps of the process, upon the program code being executed on a computer, on a processor or on a programmable hardware component.
